(19) United States
(12) Patent Application Publication
(10) Pub. No.: US 2012/0294866 A1
(43) Pub. Date: Nov. 22, 2012

(54) PHARMACEUTICAL FORMULATION FOR PROTEINS

(75) Inventors: Hanns-Christian Mahler, Basel (CH); Satya Krishna Kishore Ravuri, Birsfelden (CH)

(73) Assignee: F. HOFFMANN-LA ROCHE AG, Basel (CH)

(21) Appl. No.: 13/574,071

(22) PCT Filed: Jan. 14, 2010
(86) PCT No.: PCT/EP11/50427

§ 371 Date: Jul. 19, 2012

(30) Foreign Application Priority Data
Jan. 19, 2010 (EP) 10151021.2

(51) Int. Cl. A61K 39/395; C07D 307/62; C07C 39/06; C07C 229/60; C01D 5/00; C07C 69/88; C07C 229/26; C07C 59/265; C07C 31/26; C07C 31/08; A61K 38/00; C07C 323/58

(52) U.S. Cl. 424/141.1; 514/1.1; 424/130.1; 549/315; 568/784; 562/458; 562/556; 560/70; 562/566; 562/584; 562/559; 568/852; 568/840; 423/519.2

(57) ABSTRACT

The present invention provides a liquid pharmaceutical formulation comprising a therapeutic protein, a surfactant and at least an antioxidant selected from the group of radical scavengers, chelating agents or chain terminators.

### RELATED APPLICATIONS

This application is made under 35 U.S.C. §371 based on International Application PCT/EP2011/050427 filed on Jan. 14, 2011 and claims the benefit of priority under 35 U.S.C. §119(a) to E.P. Application No. 10151021.2 filed on Jan. 19, 2010, all of which are hereby incorporated in their entirety by reference.

### FIELD OF THE INVENTION

The present invention provides a liquid pharmaceutical formulation comprising a therapeutic protein, a surfactant and at least an antioxidant selected from the group of radical scavengers, chelating agents of chain terminators.

### BACKGROUND OF THE INVENTION

Commonly applied processes and conditions where interfacial interactions are involved like filtration pumping agitation (for example shaking or stirring), freeze/thawing and also lyophilization may lead to aggregation. Polysorbates are an important class of non-ionic surfactants used widely in protein pharmaceuticals to stabilize the proteins against interface-induced aggregation and to minimize surface adsorption of proteins (Wang W 2005. Protein aggregation and its inhibition in biopharmaceutics. Int J Pharm 289 (1-2):1-30). Polysorbates are ubiquitous to protein formulations because of their effectiveness in protecting many proteins. In fact, specifically for monoclonal antibodies (Mabs), more than 70% of the marketed formulations contain either polysorbate 20 or 80 (PS20 or PS80). The prevalent use of polysorbates is due to their high HLB numbers, low CMC values and thus very efficient surface activity at low concentrations. The mechanism of action of polysorbates in stabilizing proteins is considered to be based on their surface activity and thus interaction at interfaces in competition with a protein, though the CMC itself has not been found to the major parameter. The high affinity of polysorbates to surfaces is evident from the fact that polysorbates themselves will interact with surfaces, such as filters. Polysorbates are amphiphilic, non-ionic surfactants composed of fatty acid esters of polyoxyethylene (POE) sorbitan. Commercially available Polysorbates are chemically diverse mixtures containing mainly sorbitan POE fatty acid esters. Additionally substantial amounts of POE, sorbitan POE and Isosorbide POE fatty acid esters are present. It is known that polysorbates are prone to degradation by auto-oxidation and hydrolysis. Despite the current level of knowledge on the degradation of polysorbates (Kerwin BA 2008. Polysorbates 20 and 80 used in the formulation of protein biotherapeutics: Structure and degradation pathways. J Pharm Sci 97(8):2924-2935), the fate of polysorbates used in a parenteral protein formulation warrants a closer study in order to gain understanding on the time course as well as the mechanism of degradation under pharmaceutically relevant conditions. This is especially pertinent, since not much is known on the interaction or influence of the degraded polysorbate species on the stability of the protein.

Polysorbates are known to undergo degradation over time both in bulk and in aqueous solutions by two mechanisms a) hydrolysis; b) auto oxidation.

The degradation of polysorbate could have a potential influence on the product quality (a) by not stabilizing the protein anymore and thus having a negative influence or (b) due to a buildup of insoluble degradation products which could potentially appear as “particles” in the product over time.

Therefore, there is a need for a pharmaceutical formulation for proteins which overcomes at least in part the drawbacks of prior art pharmaceutical formulations for proteins.

### SUMMARY OF THE INVENTION

One embodiment of the invention provides a liquid pharmaceutical formulation comprising a protein, a surfactant and at least one antioxidant selected from the group of radical scavengers, chelators, chain terminators, and combinations thereof. In some embodiments, the at least one antioxidant is a radical scavenger. In some embodiments, the radical scavenger is selected from ascorbic acid, BHT, BHA, sodium sulfite, p-amino benzoic acid, glutathione and propyl gallate. In some embodiments, the protein is a therapeutic protein. In some embodiments, the protein is an antibody. In some embodiments, the antibody is a monoclonal antibody. In some embodiments, the chelator is selected from EDTA and citric acid. In some embodiments, the chelator is EDTA. In some embodiments, the chelator is citric acid. In some embodiments, the chain terminator is selected from methionine, sorbitol, ethanol and N-acetyl cysteine. In some embodiments, the surfactant is selected from the group of polysorbate and poloxamer. In some embodiments the polysorbate is polysorbate 20 or polysorbate 80.

Another embodiment of the inventions provides the use of at least one antioxidant selected from the group consisting of radical scavengers, chelators or chain terminators for prevention of surfactant degradation in a liquid pharmaceutical formulation comprising a protein. In some embodiments, the at least one antioxidant is a radical scavenger. In some embodiments, the radical scavenger is selected from ascorbic acid, BHT, BHA, sodium sulfite, p-amino benzoic acid, glutathione and propyl gallate. In some embodiments, the protein is a therapeutic protein. In some embodiments, the protein is an antibody. In some embodiments, the antibody is a monoclonal antibody. In some embodiments, the chelator is selected from EDTA and citric acid. In some embodiments, the chelator is EDTA. In some embodiments, the chelator is citric acid. In some embodiments, the chain terminator is selected from methionine, sorbitol, ethanol and N-acetyl cysteine. In some embodiments, the surfactant is selected from the group of polysorbate and poloxamer. In some embodiments the polysorbate is polysorbate 20 or polysorbate 80.

### BRIEF DESCRIPTION OF THE DRAWINGS

FIG. 1A shows the increase in peroxide content in formulations over storage time of 6 months at three different temperatures in polysorbate 20,

FIG. 1B shows the increase in peroxide content in formulations over storage time of 6 months at three different temperatures in polysorbate 80,

FIG. 2A shows the decrease in polysorbate concentration over storage time of 6 months in polysorbate 20 as measured by HPLC/ELSD method,

FIG. 2B shows the decrease in polysorbate concentration over storage time of 6 months in polysorbate 80 as measured by HPLC/ELSD method,

FIG. 3A shows Polysorbate concentration of PS20 in presence and absence of BHT/EDTA/Methioine. Excipients are: P1=polysorbate, P2=polysorbate+BHT, P3=polysorbate+EDTA, P4=polysorbate+Methionine,

FIG. 3B shows Polysorbate concentration of PS80 in presence and absence of BHT/EDTA/Methionine, Excipients are: P1=polysorbate, P2=polysorbate+BHT, P3=polysorbate+EDTA, P4=polysorbate+Methionine,

FIG. 4 shows the results of intentionally degraded polysorbate 20 in absence or presence of variety of excipients for prevention of degradation of polysorbate.

### DETAILED DESCRIPTION OF THE INVENTION

### I. Definitions

As used herein, the term “pharmaceutical formulation” (or “formulation”) means, e.g., a mixture or solution containing a therapeutically effective amount of an active pharmaceutical ingredient e.g. a polypeptide or an antibody, together with pharmaceutically acceptable excipients to be administered to a mammal, e.g., a human in need thereof.

The term “polypeptide” as used herein, refers to a polymer of amino acids, and not to a specific length. Thus, peptides, oligopeptides and protein fragments are included within the definition of polypeptide.

The term “antibody” encompasses the various forms of antibody structures including but not being limited to whole antibodies and antibody fragments. The antibody according to the invention is preferably a humanized antibody, chimeric antibody, or further genetically engineered antibody as long as the characteristic properties according to the invention are retained.

“Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g. described in Houston, J. S., Methods in Enzymol. 203 (1991) 46-96). The terms “monoclonal antibody” or “monoclonal antibody formulation” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to Clq binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to Clq binding and/or FcR binding, e.g. by “class switching” i.e. change or mutation of Fc parts (e.g. from IgG1 to IgG4 and/or IgG1/IgG4 mutation.).

The term “pharmaceutically acceptable excipient” refers to any ingredient having no therapeutic activity and having acceptable toxcicity such as buffers, solvents, tonicity agents, stabilizers, antioxidants, surfactants or polymers used in formulating pharmaceutical products. They are generally safe for administering to humans according to established governmental standards, including those promulgated by the United States Food and Drug Administration.

The term “buffer” as used herein denotes a pharmaceutically acceptable excipient, which stabilizes the pH of a pharmaceutical preparation. Suitable buffers are well known in the art and can be found in the literature. Preferred pharmaceutically acceptable buffers comprise but are not limited to histidine-buffers, citrate-buffers, succinate-buffers, acetate-buffers and phosphate-buffers or mixtures thereof. Most preferred buffers comprise citrate, L-histidine or mixtures of L-histidine and L-histidine hydrochloride. Other preferred buffer is acetate buffer. Independently from the buffer used, the pH can be adjusted with an acid or a base known in the art, e.g. hydrochloric acid, acetic acid, phosphoric acid, sulfuric acid and citric acid, sodium hydroxide and potassium hydroxide.

The term “tonicity agent” as used herein denotes pharmaceutically acceptable excipient used to modulate the tonicity of a formulation. Tonicity in general relates to the osmotic pressure of a solution usually relative to that of human blood serum. The formulation can be hypotonic, isotonic or hypertonic. A formulation is typically preferably isotonic. An isotonic formulation is liquid or liquid reconstituted from a solid form, e.g. from a lyophilized form and denotes a solution having the same tonicity as some other solution with which it is compared, such as physiologic salt solution and the blood serum. Suitable tonicity agents comprise but are not limited to salts, amino acids and sugars. Preferred tonicity agents are sodium chloride, trehalose, sucrose or arginine.

The “tonicity” is a measure of the osmotic pressure of two solutions separated by a semipermeable membrane. Osmotic pressure is the pressure that must be applied to a solution to prevent the inward flow of water across a semi-permeable membrane. Osmotic pressure and tonicity are influenced only by solutes that cannot cross the membrane, as only these exert an osmotic pressure. Solutes able to freely cross the membrane do not affect tonicity because they will always be in equal concentrations on both sides of the membrane.

The term “amino acid” in context with tonicity agent or stabilizer, denotes a pharmaceutically acceptable organic molecule possessing an amino moiety located at a-position to a carboxylic group. Examples of amino acids include arginine, glycine, ornithine, lysine, histidine, glutamic acid, asparagic acid, isoleucine, leucine, alanine, phenylalanine, tyrosine, tryptophane, methionine, serine, proline. Preferred amino acid in context with tonicity agent or stabilizer is arginine, tryptophane, methionine, histidine or glycine.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra-, penta- and so forth saccharide. In contrast to polysaccharides the monosaccharides and oligosaccharides are water soluble. Examples of oligosaccharides include sucrose, trehalose, lactose, maltose and raffinose. Preferred sugars are sucrose and trehalose.

The term “surfactant” as used herein denotes a pharmaceutically acceptable excipient which is used to protect protein formulations against mechanical stresses like agitation and shearing. Examples of pharmaceutically acceptable surfactants include poloxamers, polysorbates, polyoxyethylene alkyl ethers (Brij), alkylphenylpolyoxyethylene ethers (Triton-X) or sodium dodecyl sulphate (SDS). Preferred surfactants are polysorbates and poloxamers.

As used herein, the term “polysorbate” refers to oleate esters of sorbitol and its anhydrides, typically copolymerized with ethylene oxide. Preferred polysorbates are Polysorbate 20 (poly(ethylene oxide) (20) sorbitan monolaurate, Tween 20) or Polysorbate 80 (poly(ethylene oxide) (80) sorbitan monolaurate, Tween 80).

The term “poloxamer” as used herein refers to non-ionic triblock copolymers composed of a central hydrophobic chain of polypropylene oxide) (PPO) flanked by two hydrophilic chains of poly(ethylene oxide) (PEO), each PPO or PEO chain can be of different molecular weights. Poloxamers are also known by the trade name Pluronics. Preferred Poloxamer is Poloxamer 188, a poloxamer wherein the PPO chain has a molecular mass of 1800 g/mol and a PEO content of 80% (w/w).

The term “antioxidant” denotes pharmaceutically acceptable excipients, which prevent α-idation of the active pharmaceutical ingredient. This includes chelating agents, reactive oxygen scavengers and chain terminators. Antioxidants comprise but are not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine.

### EXAMPLES

### Example 1

### Long Term Studies

Test Study 1

Formulations were prepared using 20 mM H is/His.HCl (SA Ajinomoto Omnichem NV, Louvain-la-Neuve, Belgium) at pH 6 with 240 mM trehalose and either 0.02% or (w/v) of PS 20 or PS 80, in the absence of protein (“placebo”). 0.001% BHT (Fluka Chemmie AG, Steinenheim), 0.01% EDTA (Fluka Chemmie AG, Steinenheim), 10 mM, Methionine (SA Ajinomoto Omnichem NV, Louvain-la-Neuve, Belgium) were added to the formulations. The formulations were filtered using 0.22 μm Millex GV (PVDF) syringe filter units (Millipore, Bedford, Mass., USA) and aseptically filled up to 2.4 mL in sterilized standard 6 mL Ø 20 mm type I clear glass injection vials (Schott form a vitrum AG, St. Gallen, Switzerland) and closed with Teflon® coated injection stopper (Dalkyo Seiko, Tokyo, Japan) and sealed with an aluminium crimp cap. Vials were stored at 5° C., 25° C. and 40° C. Samples were analyzed at time points spread over 3 months.

Test Study 2

Formulations were prepared using 20 mM H is/His.HCl (SA Ajinomoto Omnichem NV, Louvain-la-Neuve, Belgium) at pH 6 with 240 mM trehalose and either 0.02% or (w/v) of PS 20 in the absence of protein (“placebo”). The following anti-oxidants were tested: 0.005% BHT (Fluka Chemmie AG, Steinenheim, Switzerland), 0.1% EDTA (Fluka Chemmie AG, Steinenheim, Switzerland), 20 mM, Methionine (SA Ajinomoto Omnichem NV, Louvain-la-Neuve, Belgium), 20 mM, Citric acid (Fluka Chemmie AG, Steinenheim, Switzerland), 0.5% Ascorbic acid (Acros organics, Geel Belgium), 0.1% glutathione (Fluka Chemmie AG, Steinenheim, Switzerland), 0.2% sodium sulfite (Merck KGaA, Darmstadt, Germany), 0.5% Sorbitol (Fluka Chemmie AG, Steinenheim, Switzerland), 0.5% N-acetyl cystine (Fluka Chemmie AG, Steinenheim, Switzerland), 0.01% propyl gallate (Fluka Chemmie AG, Steinenheim, Switzerland), 0.01% p-amino benzoic acid (Fluka Chemmie AG, Steinenheim, Switzerland), and Poloxamer 188 were added to the formulations. The formulations were spiked with either 300 ppm H₂O₂ or 100 ppm FeCl₂. The formulations were filtered using 0.22 μm Millex GV (PVDF) syringe filter units (Millipore, Bedford, Mass., USA) and aseptically filled up to 2.4 mL in sterilized standard 6 mL Ø 20 mm type I clear glass injection vials (Schott form a vitrum AG, St. Gallen, Switzerland) and closed with Teflon® coated injection stopper (Daikyo Seiko, Tokyo, Japan) and sealed with an aluminium crimp cap. Vials were stored at 25° C. and 40° C. Samples were analyzed at time points spread over 3 weeks

Polysorbate Quantification

Quantification of polysorbate concentration in formulations was done either using an HPLC/ELSD based method or by a fluorescence micelle method.

(a) HPLC/ELSD Method

The HPLC/ELSD method was based on the one described by Hewitt et at (Hewitt D, Zhang T, Kao YH 2008. Quantitation of polysorbate 20 in protein solutions using mixed-mode chromatography and evaporative light scattering detection. J Chromatogr A 1215(1-2):156-160) to analyze polysorbate in the formulations. A 30 μm mixed-mode column Oasis MAX from Waters was utilized. The polysorbate peak area was determined and compared to a calibration curve. In order to exclude potential assay interference the calibration standard contained all excipients which were present in the samples.

(b) Fluorescence Micelle Method

A fluorescent micelle assay was used to determine the concentration of polysorbate in the extraction samples. The assay is based on the uptake of fluorescent dye N-phenyl-1-naphtylamine (NPN) into the hydrophobic core of polysorbate micelles. NPN has a low fluorescent quantum yield in an aqueous environment whereas a high yield is observed in a nonpolar setting. The test was set up as flow injection assay (FIA) using a Waters 2695 HPLC (Milford, Mass.) connected via a 750 mL knitted reaction coil (Dionex, Sunnyvale, Calif.) to a Waters 474 fluorescence detector. The fluorescence detector was set to an excitation wavelength of 350 nm and an emission wavelength of 420 nm. The mobile phase consists of 0.15 M sodium chloride, 0.05 M TRIS, pH 8.0, 5% acetonitrile, 15 ppm Brij35 and 5.0 mM NPN(N-phenyl-1-napthylamine). For quantitation, the polysorbate peak area was determined and compared to a calibration curve. In order to exclude potential assay interference the calibration standard contained all excipients which were present in the samples.

Peroxide Determination

Peroxide determination was performed with a commercially available peroxide quantification kit PeroXOquant from Thermo Fischer based on the FOXII assay (Ha E, Wang W, Wang YJ 2002. Peroxide formation in polysorbate 80 and protein stability. J Pharm Sci 91(10):2252-2264) which relies upon the rapid hydroperoxide-mediated oxidation of Fe²⁺ to Fe³⁺ under acidic conditions and complexes with xylenol orange which absorbs strongly at 560 nm. Formulations without protein were used for peroxide determination.

Results:

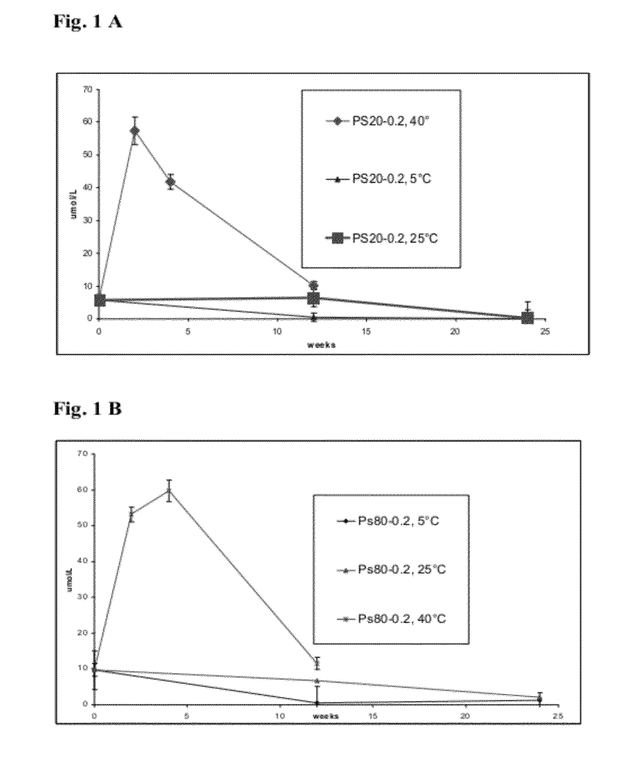

The peroxide concentration was found to increase in formulation solution (FIGS. 1A and B). Although this increase has been noticed previously by others in bulk and aqueous solutions, no reports of the same are noted in pharmaceutically relevant conditions.

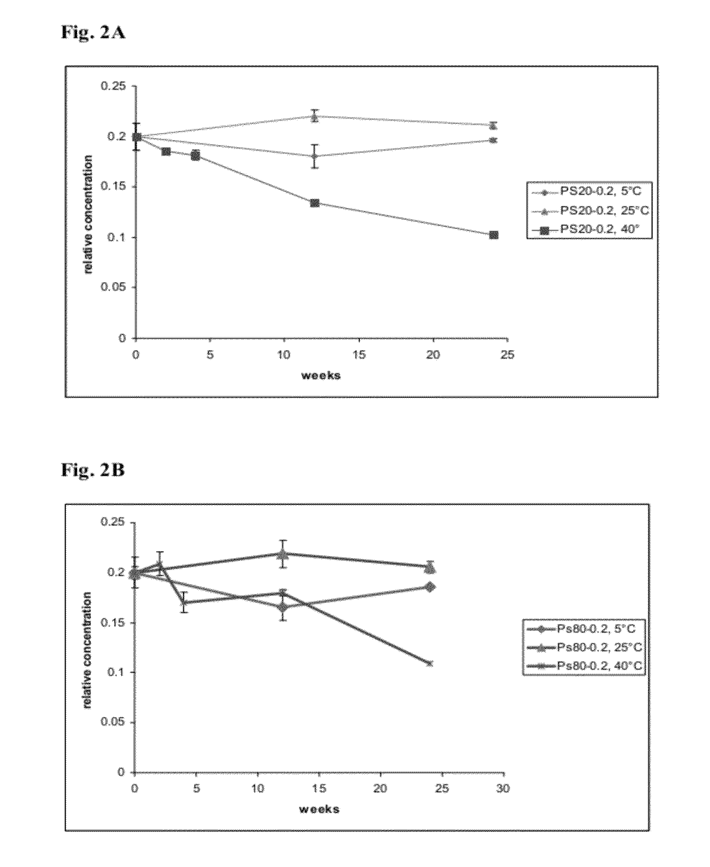

The polysorbate concentration was found to decrease in formulation solution over time and more pronounced with higher temperatures (FIGS. 2 A and B).

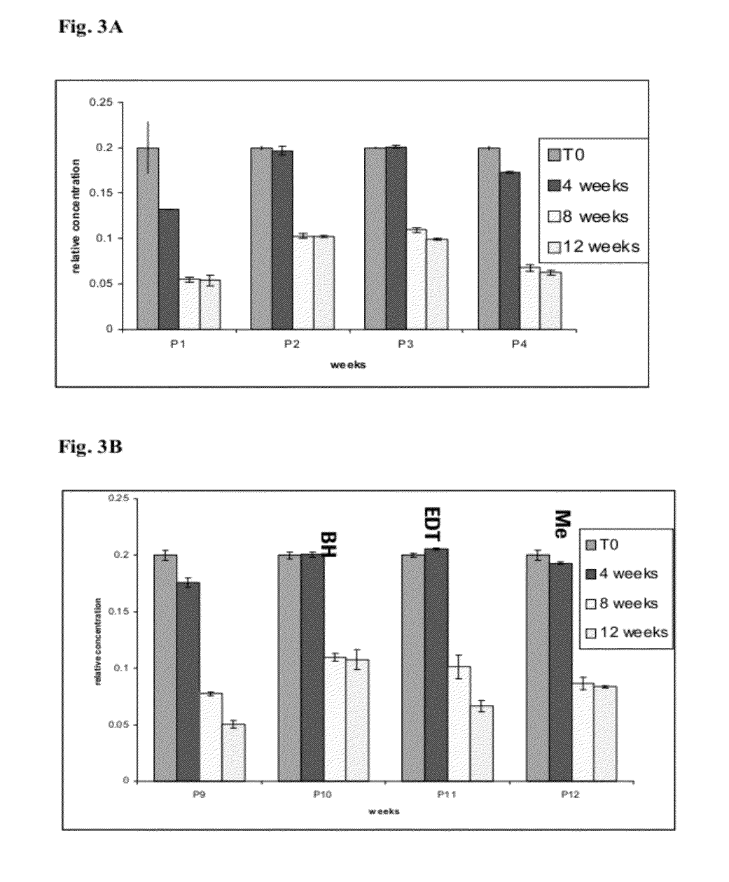

Inclusion of an additional component may improve the stability of polysorbate in aqueous formulations when compared to those without. This was established by testing the polysorbate content in formulations spiked with BHT, with Methionine and with EDTA. It was clear that addition of these has a positive effect in minimizing degradation of polysorbates (FIGS. 3A and B).

The following components were further screened for their potential to minimize polysorbate degradation:

TABLE 1

| Type of additive | Concentration |
|---|---|
| no additive | |
| EDTA | 0.10% |
| citric acid | 20 mM |
| Methionine | 20 mM |
| ascorbic acid | 0.50% |
| glutathione | 0.10% |
| sodium sulfite | 0.2% |
| Sorbitol | 0.50% |
| N-acetyl-cysteine | 0.50% |
| BHT | 0.005% |
| propyl gallate | 0.01% |
| p-aminobenzoic acid | 0.01% |
| Ethanol | 0.01% |

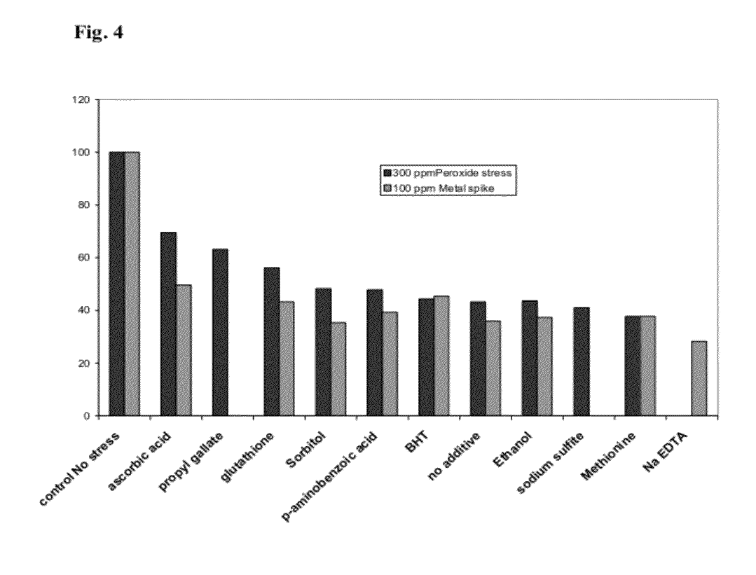

The antioxidants tested were broadly in the category of chelators (eg EDTA, Citric acid), reactive oxygen scavengers (eg Ascorbic acid, BHT, sodium sulfite, p-amino benzoic acid, glutathione, propyl gallate) and chain terminators (eg Methionine, sorbitol, ethanol and N-acetyl cysteine) (FIG. 4).

The formulations were tested against aggressive oxidation stress conditions inducing polysorbate degradation, namely 300 ppm H₂O₂ and 100 ppm FeCl₂.

It was again confirmed that with increasing temperature and with longer time, the polysorbate degradation was more pronounced.

Especially radical scavengers seem to be playing a very important role as compared to chelators and chain terminators and all above excipients have shown improvement, i.e. have minimized polysorbate degradation.

1. A liquid pharmaceutical formulation comprising a protein, a surfactant and at least one antioxidant selected from the group of radical scavengers, chelators or chain terminators.

2. The liquid pharmaceutical formulation of claim 1, wherein the at least one antioxidant is selected from the group of radical scavengers.

3. The liquid pharmaceutical formulation of claim 2, wherein the radical scavenger is selected from ascorbic acid, BHT, BHA, sodium sulfite, p-amino benzoic acid, glutathione and propyl gallate.

4. The liquid pharmaceutical formulation of claim 1 to 3, wherein the protein is a therapeutic protein, preferably an antibody, more preferably a monoclonal antibody.

5. The liquid pharmaceutical formulation of claim 1 to 4, wherein the chelator is selected from EDTA and citric acid.

6. The liquid pharmaceutical formulation of claims 1 to 5, wherein the chain terminator is selected from methionine, sorbitol, ethanol and N-acetyl cysteine.

7. The liquid pharmaceutical formulation of claims 1 to 7, wherein the surfactant is selected from the group of polysorbate and poloxamer.

8. The liquid pharmaceutical formulation of claim 7, wherein the polysorbate is polysorbate 20 or polysorbate 80.

9. Use of an antioxidant selected from the group consisting of radical scavengers, chelators or chain terminators for prevention of surfactant degradation in a liquid pharmaceutical formulation comprising a protein.

10. The use of an antioxidant of claim 9, wherein the radical scavenger is selected from ascorbic acid, BHT, sodium sulfite, p-amino benzoic acid, glutathione and propyl gallate.

11. The use of an antioxidant of claim 9 or 10, wherein the chelator is selected from EDTA and citric acid.

12. The use of an antioxidant of claims 9 to 11, wherein the chain terminator is selected from the group of methionine, sorbitol, ethanol and N-acetyl cysteine.

13. The use of an antioxidant of claims 9 to 12, wherein the protein is a therapeutic protein, preferably an antibody, more preferably a monoclonal antibody.

14. The use of an antioxidant of claims 9 to 13, wherein the surfactant is selected from the group of polysorbate and poloxamer.

15. The use of an antioxidant of claim 14, wherein the polysorbate is polysorbate 20 and/or polysorbate 80.
